(19) United States
(12) Patent Application Publication
(10) Pub. No.: US 2023/0346862 A1
(43) Pub. Date: Nov. 2, 2023

(54) CANCER ERADICATING - BIO-NANOPARTICLES (CE-BNP)

(71) Applicant: ATHANOR BIOSCIENCES, INC., HALETHORPE, MD (US)

(72) Inventors: HOSSEIN A. GHANBARI, POTOMAC, MD (US); MICHAEL S. LEBOWITZ, PIKESVILLE, MD (US)

(21) Appl. No.: 18/310,788

(22) Filed: May 2, 2023

Related U.S. Application Data
(60) Provisional application No. 63/337,351, filed on May 2, 2022.
(60) Provisional application No. 63/338,628, filed on May 5, 2022.

(51) Int. Cl. A61K 35/76; C12N 7/00; A61K 45/06; C07K 16/40; C07K 14/21; A61P 35/00; C07K 16/30; A61P 13/08; A61P 35/02

(52) U.S. Cl. CPC A61K 35/76; C12N 7/00; A61K 45/06; C07K 16/40; C07K 14/21; A61P 35/00; C07K 16/30; A61P 13/08; C07K 16/3069; A61P 35/02; C12N 2795/00021; C07K 2317/622; C12N 2795/00043; C12N 2795/00052

(57) ABSTRACT

Cancer eradicating engineered bacteriophage are described that can display a high copy number of a targeting polypeptide that can bind a surface antigen of a cancer cell. The bacteriophage can also display a high copy number of a cancer therapy, one or more of a drug, a toxin, an inhibitor, a radionuclide, etc. The targeting polypeptides and the cancer therapies can be directly or indirectly fused to coat proteins of the phage. The engineered phage can exhibit high avidity for cancer cells and can deliver a large dose of a cancer therapy per particle to the cell.

### CROSS REFERENCE TO RELATED APPLICATION

This application claims filing benefit of U.S. Provisional Patent Application Ser. No. 63/337,351 having a filing date of May 2, 2022, entitled, “Bacteriophage-Based Cancer Therapies That Target Lactic Dehydrogenase,” and claims filing benefit of U.S. Provisional Patent Application Ser. No. 63/338,628, having a filing date of May 5, 2022, entitled, “Cancer Eradicating—Bio-Nanoparticles (CE-BNPS), both of which are incorporated herein by reference in their entirety for all purposes.

### BACKGROUND

Cancer is a devastating disease in which an individual's cells acquire a series of genetic mutations and become transformed into a variant cell type that is capable of uncontrolled growth and proliferation as well as metastatic potential—the ability to break away from the tissue of origin and spread to other parts of the body. There are many different types of cancers and these can be categorized by the tissue of origin, by the resulting phenotype and morphological features and/or by the molecular genetic changes that underlie the transformative process. Cancerous cells often grow into tumors of varying sizes and metastasize to other sites within the body. If left unchecked, this uncontrolled growth and metastasis can lead to death due to physical disruption of normal organ function, depletion of nutrients and resources, release of toxic agents, disruption of normal physiology and/or interference with normal cellular communication.

Antibody-based cancer therapies cover a broad spectrum of different approaches to specifically target cancer cells. In their simplest modality, antibody-based therapies utilize antibodies that have been selected to specifically bind a tumor-associated antigen to identify and bind to cancer cells. The antibody might in turn inhibit or activate specific signaling pathways within a cell resulting in an alteration in tumor cell function, ideally leading to cell death. Alternatively, the antibody could simply bind to the surface of the tumor cells and activate antibody-dependent cellular cytotoxicity (ADCC) or complement-dependent cytotoxicity (CDC). ADCC is a process in which an immune effector cell, such as an NK cell, is recruited to the target cell, is activated, and subsequently induces apoptosis of the target tumor cell. CDC is a process by which the antibodies activate the complement cascade resulting in the formation of the membrane attack complex (MAC) and target cell lysis.

Antibodies generally consist of antigen binding domains and constant regions. The antigen binding domains are highly variable in sequence and specific to the target antigen. The constant regions interact with immune effector cells and/or complement. Due to the exquisite specificity of antibodies for their antigens, they have been designed for use as a means of specific delivery of secondary agents to targeted cells, including cancer cells.

One class of antibody-based cancer therapies provides for attachment of a molecular cancer treatment to the antigen binding domains of antibodies. Examples of such therapies include immunotoxins, antibody-drug conjugates (ADCs), and radioimmunotherapy (RIT). Immunotoxins are engineered proteins that combine a targeting domain such as an antibody or antibody fragment (scFv) or a protein ligand (e.g., IL-2) with a toxic protein such as a bacterial toxin. ADCs link an antibody-based targeting domain with a drug (e.g., a non-proteinaceous drug) designed to destroy the targeted cell type. RIT is an approach in which an antibody is modified to chelate radioactive nuclides to provide targeted delivery of a radionuclide that can kill the targeted cell.

While such therapies have provided improvement in cancer treatment, there are a number of drawbacks that limit the utility and efficacy of cancer immunotherapies. Specifically, issues arise regarding the affinity and specificity of the targeting moiety, potency, ability to deliver significant amounts of the payload to the cancer cell, immunogenicity of agents resulting in rapid clearance, inability to give multiple doses, and difficulties and expense related to manufacture of these agents. For instance, the ratio of payload material (e.g., drug) to antibody is generally only around 3 per antibody and as such, and particularly when using a low potency drug, a large amount of the conjugate will need to be endocytosed by the cell to yield significant results. A recent study demonstrated a linear relationship between HER2 levels in cells and drug delivery by a model ADC, trastuzumab-valine-citrulline-monomethyl auristatin (T-vc-MMAE) (Sharma S, Li Z, Bussing D, Shah D K. 2020. Drug Metab. Dispos. 48(5):368-77). The study observed ADC efficacy, as measured primarily by intracellular MMAE release, in a series of 4 cell lines that expressed as little as about 10,000 and as many as about 800,000 HER2 surface receptors. Two doses of ADC were tested, 1 nM and 10 nM, and a linear relationship was observed between receptor level and drug delivery. However, at the lowest receptor abundance level (about 10,000 HER2/cell), the rate of drug delivery could not be determined due to the “negligible” levels of receptor and this was effectively dose independent as receptors were already saturated at 1 nM T-vc-MMAE. Unfortunately, the requirement of significant receptor abundance precludes the targeting of a large number of possible tumor-specific antigens because of their relatively low surface expression levels. This is despite the fact that these receptors are “overexpressed” in tumor cells as in this case “overexpression” is a relative term compared to levels in non-cancer cells.

Bacteriophage (or more simply phage) are viruses that infect bacterial cells. These viruses consist of a protein coat which encapsulates a DNA or RNA genome. When phage infect a bacterial cell, they can coopt the host bacterial system to produce large numbers of phage copies and ultimately lyse the bacterial cell, releasing the new phage to the surrounding environment.

It has been demonstrated that phage can be used to display large numbers of peptide or protein fragments. For example, phage display systems have been used to map the epitopes of antibodies and to identify single chain fragments of antibodies (scFv) that bind to specific antigens. These phage display systems gain their selection power from the ability to display many copies of a protein on the surface of the phage. By way of example, when using bacteriophage λ, the displayed protein is often engineered as an extension of the phage gpD coat protein. 400 copies of the gpD protein are used by the phage to construct its coat and as such up to 400 copies of the requisite protein can be displayed on the phage surface. In the case of bacteriophage λ, proteins of up to 300 amino acids can be displayed without disrupting the ability of the phage coat to form. This ability of phage to present on their surfaces large numbers of proteins or protein fragments qualifies them as bio-nanoparticles (BNPs).

What are needed in the art are improved immunotherapy materials and methods that can address limitations of known systems and methods.

### SUMMARY

According to one embodiment, disclosed is an engineered multivalent, multiplexed bacteriophage that includes multiple fusion coat proteins. A first fusion coat protein can include an exogenous targeting polypeptide directly or indirectly fused to a coat protein of the bacteriophage. This exogenous targeting polypeptide can include a binding sequence that binds a cancer cell surface antigen. A second fusion coat protein can carry a proteinaceous or non-proteinaceous cancer therapy. For instance, a second fusion coat protein can include an exogenous polypeptide that includes the cancer therapy, e.g., a proteinaceous toxin, drug, or inhibitor. The second fusion coat protein can optionally include an exogenous polypeptide that serves as a linker to bind a cancer therapy to the coat protein. In one embodiment, the second fusion coat protein can include a linker that binds a non-proteinaceous cancer therapy, e.g., a small molecule drug or radionuclide, to the coat protein of the bacteriophage. Beneficially, the engineered bacteriophage can include multiple copies of both the first and second fusion coat proteins. The engineered bacteriophage can also be free of nucleic acids that encode the exogenous polypeptides, while still carrying its own genome.

Therapeutic compositions are also described that include an engineered multivalent, multiplexed bacteriophage as described in conjunction with a delivery system.

Also disclosed are methods for forming the multivalent, multiplexed engineered bacteriophage. A method can include transfecting a bacterial cell with one or more expression plasmids and also infecting the bacterial cell with a phage. The expression plasmid(s) can include a first hybrid nucleic acid sequence that encodes the first fusion coat protein designed for targeting the bacteriophage to a cancer cell and a second nucleic acid sequence that encodes the second fusion coat protein designed to carry the cancer therapy (the therapy itself and/or a linker). The expression plasmid can also include regulatory sequences such that following the transfection, the fusion coat protein is transiently expressed by the bacterial cell. Upon the infection and the transfection, an engineered phage can be produced by the bacterial cell that includes multiple copies of both of the fusion coat proteins. Depending upon the nature of the cancer therapy, a method can also include conjugating a non-proteinaceous cancer therapy to the second fusion coat protein via the linker. Thus, a method can form a multivalent, multiplexed phage with high affinity and binding avidity for the targeted cancer cells as well as large doses of the cancer therapy.

### BRIEF DESCRIPTION OF THE FIGURES

A full and enabling disclosure of the present subject matter, including the best mode thereof to one of ordinary skill in the art, is set forth more particularly in the remainder of the specification, including reference to:

FIG. 1, which schematically illustrates one embodiment of a multivalent, multiplexed immunotherapeutic bacteriophage as described herein; and

FIG. 2, which graphically illustrates effectiveness of multivalent, multiplexed engineered bacteriophage as described herein against a pancreatic cell line and a prostate cancer cell line.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment. Moreover, it is noted that all US patent and patent applications mentioned herein are incorporated by reference thereto.

In general, the present disclosure is directed to formation and use of engineered multivalent, multiplexed bacteriophage, i.e., BNPs that can overcome issues of existing immunotherapies such as those discussed above and can function as cancer eradicating—bio-nanoparticles (CE-BNP).

Disclosed engineered bacteriophage (also referred to as CE-BNP throughout this disclosure) can display high copy numbers of a targeting polypeptide that can bind a surface antigen of a targeted cancer cell, and as such can achieve high affinity/avidity for the targeted cancer cell. Beneficially, a single phage can include multiple copies of a targeting polypeptide as well as, in some embodiments, multiple different targeting polypeptides. The targeting polypeptides can be directly or indirectly fused to coat proteins of the phage. In some embodiments multiple targeting polypeptides of a single phage can be directly or indirectly bonded to one another, for instance as multiple targeting polypeptides directly or indirectly (e.g., via linking polypeptides) bonded to a terminal end of a single phage coat protein. Such capabilities can further increase the specificity of the engineered phage for the targeted cancer cell and by targeting two or more low abundant antigens with inclusion of two or more different targeting polypeptides can increase the available number of target sites.

Disclosed materials can also display high copy numbers of one or more cancer therapies and as such can deliver a large dose of a cancer therapy per particle to the cell. This can provide for increased efficiency of cancer therapy delivery given that with each endocytosed particle hundreds of copies of the cancer therapy can be delivered. Moreover, by supplying multiple different cancer therapies in high copy number, more than one approach for cancer cell destruction can be delivered.

Due to the large number of coat proteins available on a phage, the engineered phage can be further supplied with polypeptides that can provide other uses, e.g., labeling, to control immunogenicity against the phage during use by downregulating presentation by antigen presenting cells (APCs), or any other useful purpose.

In addition to the above advantages, disclosed engineered phage can also be free of nucleic acid encoding the exogenous polypeptide(s), and as such, there is no concern of transfer of foreign DNA or RNA to a subject, which can be particularly beneficial in cancer treatments. In some embodiments, the DNA and/or RNA of the phage genome encased within the capsid of the engineered phage, which will still be present in the bacterially produced product, can include a modification from wild-type phage, e.g., a stop codon that can prevent further replication of the phage following production, an insert to control immunogenicity, etc.

The use of phage as BNPs as compared to other synthetic nanoparticles or bio-nanoparticles is especially advantageous. Phage are simple to genetically engineer and are easy to purify. Furthermore, phage can be produced at extremely high yield in readily available biofermenters. As such, the development and manufacturing processes can be rapid and highly cost-effective. In vivo, phage are known to have long half-lives and their size allows for easy tissue penetration. In general, phage have demonstrated only low levels of immunogenicity in mammals, including humans, likely due to early exposure and partial acquired immune tolerance due to the abundance of phage in the natural environment. Moreover, recent developments in phage formation have provided phage as potential starting materials for disclosed CE-BNP that are even less immunogenic that previously known phage due to genetic modification of certain phage proteins (e.g. Merril C R, et al. 1996 Proc. Natl. Acad. Sci. USA, 93:3188-92), contain a conditional expression of a wildtype phage protein (e.g. phage λ gpD as in Nicastro J, et al. 2013 Appl Microbiol Biotechnol 97:7791-804) and/or incapable of further reproduction, which can be advantageous in some embodiments. Additionally, as the phage are BNP, they can optionally be irradiated prior to use to prevent any potential infectivity to a subject.

Phage that may be modified to provide disclosed CE-BNP may be any bacteriophage known to those skilled in the art, including but not limited to λ, M13, T4, T7, φX174. The coat protein to which the exogenous polypeptides are fused in forming a fused coat protein will depend on the type of phage employed as well as the number of exogenous polypeptides desired for each phage. For λ phage, the gpD, gpE or gpC proteins can be used, with over 400 copies of each of the gpD and gpE in each phage. For M13 phage, the pVIII, pIII, pVI, pVII or pIX proteins can be used. For T4 phage, the gp23 or gp24 proteins can be used and for T7 phage the gp10A or gp10B proteins can be used. For phage φX174, the gpF or gpG proteins can be used. The numbers of copies of each of these proteins within the specific phage type varies from tens of copies to hundreds of copies, as is known. For example, bacteriophage λ have two major capsid proteins, gpD and gpE, which are incorporated in over 400 copies each in the phage.

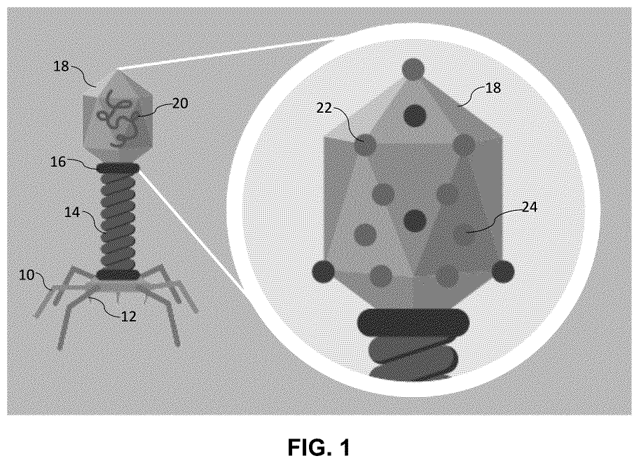

FIG. 1 schematically illustrates one embodiment of an engineered multivalent, multiplexed bacteriophage designed for cancer eradication. As illustrated, a bacteriophage can include typical bacteriophage components including tail fiber 10, spikes 12, and a sheath 14. A collar 16 typically separates the sheath 14 from the capsid head 18, which encases the bacteriophage endogenous RNA or DNA genome 20. The capsid head 18 is formed from a plurality of coat proteins, e.g., gpD, gpE and gpC coat protein in the case of bacteriophage λ. Cancer eradicating bacteriophage as disclosed herein can include two or more different fusion coat proteins 22, 24 in the capsid head 18 that include an exogenous polypeptide at a terminal end of a coat protein, which can be either or both of an N-terminus or C-terminus of the coat protein. One of the fusion coat proteins 22 can include an exogenous polypeptide that incorporates a targeting polypeptide configured to specifically bind a cancer cell surface antigen. Another of the fusion coat proteins 24 can include an exogenous polypeptide that can carry a proteinaceous or non-proteinaceous cancer therapy.

As utilized herein, the term “exogenous” refers to a material that originates external to and is not found as a component of either the phage or the bacterial cell that are used to produce the CE-BNP. As utilized herein, the term “polypeptide” generally refers to a polymeric molecule including two or more amino acid residues, which can include natural and synthetic amino acids as well as combinations thereof and includes proteins as well as fragments. As utilized herein, the term “fragment” generally refers to a continuous part of a full-length protein, with or without mutations, which is separate from and not in the context of a full length protein. A fragment may be a structural/topographical or functional subunit of a full length protein. In some embodiments, a fragment can have an amino acid sequence of about 15 or more amino acids, or about 20 or more amino acids of the parent full-length surface protein.

The ability to display large copy numbers of both the targeting polypeptide and the cancer therapy on the surface of a phage allows for improved effectiveness. Simply put, when a cancer cell and a CE-BNP interact, the existence of multiple binding points can greatly enhance the overall strength of the interaction (avidity). By way of example, when using bacteriophage λ, the displayed exogenous targeting polypeptide can be engineered as an extension of the phage gpD coat protein and the cancer therapy can be carried by the gpE coat protein. 400 copies of both the gpD and gpE proteins are used by the phage to construct its coat and as such, by utilizing fusion gpD proteins to carry the targeting polypeptide and fusion gpE proteins to carry the cancer therapy up to 400 copies of the exogenous targeting polypeptide and up to 400 copies of the cancer therapy can be displayed on the phage surface. Moreover, the exogenous polypeptides of a fusion coat protein can be quite large without disrupting the ability of the phage to form. For instance, in the case of bacteriophage λ, relatively large polypeptides (300 amino acids or more) or multiple smaller polypeptides that are the same or different from one another can be ligated to one another and included in a single fusion protein without disrupting the ability of the phage coat to form.

In general, exogenous polypeptide sequences chosen for inclusion in a fusion coat protein for use as a targeting agent, a cancer therapy, a linker, or any other use, may be derived from any source and can include complete proteins, protein fragments, mutants, or homologues thereof. In one embodiment a multivalent, multiplexed bacteriophage can be engineered that can include multiple different fragments (or homologues thereof) of a single protein, for instance, when the natural protein of interest is large and incorporation of the entire protein sequence in a single fusion coat protein could interfere with bacteriophage formation. As utilized herein, the term “homologue” generally refers to a nucleotide or polypeptide sequence that differs from a reference sequence by modification(s) that do not affect the overall functioning of the sequence. For example, when considering polypeptide sequences, homologues include polypeptides having substitution of one amino acid at a given position in the sequence for another amino acid of the same class (e.g., amino acids that share characteristics of hydrophobicity, charge, pK or other conformational or chemical properties, e.g., valine for leucine, arginine for lysine, etc.). Homologues can include one or more substitutions, deletions, or insertions, located at positions of the sequence that do not alter the conformation or folding of a polypeptide to the extent that the biological activity of the polypeptide is destroyed. Examples of possible homologues include polypeptide sequences and nucleic acids encoding polypeptide sequences that include substitution of one non-polar (hydrophobic) residue such as isoleucine, valine, leucine or methionine for another; the substitution of one polar (hydrophilic) residue for another such as between arginine and lysine, between glutamine and asparagine, or between threonine and serine; the substitution of one basic residue such as lysine, arginine or histidine for another; the substitution of one acidic residue, such as aspartic acid or glutamic acid for the another; or the use of a chemically derivatized residue in place of a non-derivatized residue, as long as the homolog displays substantially similar biological activity to the reference sequence.

In one embodiment, exogenous targeting polypeptides displayed on a phage surface may be derived from an antibody that specifically recognizes and binds one or more epitopes of a cancer cell surface antigen. These displayed polypeptides may correspond to antibodies or any derivative thereof including but not limited to single-chain antibodies (scFv) and/or nanobodies (VH or VHH sequences). The term antibodies and antibody fragments as utilized herein is intended to incorporate all antibody types derived from any species including but not limited to humans, rodents, livestock, camelids, fowl, etc. The sequences of these antibodies and/or antibody fragments may be modified such that potential human B and/or T-cell epitopes have been mutated to reduce potential immunogenicity as is known to one skilled in the art. Alternatively, proteins or protein fragments that serve as binding partners or ligands for a tumor associated surface antigen may be used.

Cancer cells that can be targeted by disclosed materials are not particularly limited, and can include both solid and hematological cancers. Exemplary cancer types that can be targeted by disclosed CE-BNPs can include, without limitation, prostate cancers, multiple myeloma, pancreatic cancers, liver cancers, bile duct cancers, brain cancers, breast cancers, colon cancers, ovarian cancers, lung cancers, leukemias, etc.

In general, the binding partner of an exogenous targeting polypeptide of a CE-BNP can be surface expressed on the targeted cancer cell and capable of being endocytosed upon engagement by the targeting polypeptide. Furthermore, there can generally be suitable amounts of the binding partner expressed on the cell surface in order that relatively large quantities of the cancer therapy can be delivered to the cell upon binding. Alternatively, and as discussed previously, a targeted cell can include a lesser number of a single binding partner, and the CE-BNP can include multiple different targeting polypeptides such that the total number of all binding partners for all targeting polypeptides of the CE-BNP can be greater. This has the advantage of increasing both the specificity to the targeted cancer cell as well as the overall affinity to the targeted cancer cell, especially when one or more tumor associated surface antigens are low abundance on the targeted cell. In any case, the exogenous targeting polypeptide(s) of the CE-BNP, e.g., an antibody or derivative thereof (single chain antibody, scFv, etc.), can afford specificity to the targeted binding partner of a cancer cell and can bind with a relatively high affinity to the cell, with endocytosis of the CE-BNP following the binding.

In one embodiment, different targeting polypeptides of a bacteriophage can specifically bind a single protein target, e.g., different peptide sequences of a single surface protein of a cancer cell. For example, a first exogenous polypeptide can include a targeting polypeptide specific for a first epitope of a cancer cell surface protein and a second, different exogenous polypeptide can include a second targeting polypeptide specific for a second epitope of the same surface protein. In one embodiment, different exogenous targeting polypeptides of a bacteriophage can specifically bind different surface proteins of the same cancer cell type. Of course, any combination of cancer cell targeting polypeptides are encompassed herein as well.

Targeting polypeptides can include antibodies or antibody fragments (e.g. scFv) that are known to bind to a tumor specific surface antigen such as, and without limitation to, aspartyl β-hydroxylase (ASPH), prostate specific membrane antigen (PSMA), B-cell maturation antigen (BCMA), melanoma associated antigen (MAGE), NY-ESO1, carcinoembroyonic antigen (CEA), human epidermal growth factor receptor 2 (HER2), CD33, nectin-4, CD30, DCD22, CD79b, TROP2, etc. However, while a tumor specific surface antigen can be targeted in one embodiment, any tumor associated surface antigen can be targeted by disclosed CE-BNP.

PSMA and BCMA are well known tumor targets representing both a solid tumor antigen and a hematological cancer antigen. Clinical applications of these targets include treatment of recurrent prostate cancer and multiple myeloma, respectively. ASPH is a low abundant tumor antigen with broad tumor expression across 20 different cancer types including both solid and hematological cancers. ASPH has been previously used as a target for an anti-cancer vaccine that entered human phase II clinical trials and has also been demonstrated as a promising target for a traditional ADC approach in patient-derived xenograft (PDX) models of pancreatic cancer. ASPH is an enzyme that hydroxylates aspartate or asparagine residues present in EGF-like domains of certain proteins including NOTCH, JAGGED, vimentin, ADAM10/17, and ADAM12/15. The normal function of the enzyme appears to be regulation of signaling pathways during embryonic development and in the case of the NOTCH/JAGGED interaction, the hydroxylation of the EGF domains results in increased downstream signaling. In cancer cells, ASPH has been shown to be translocated to the cellular surface from where it is normally located. This surface re-localization is noteworthy because it allows for, and in fact advocates the use of, disclosed immunotherapies that access the cell surface.

Antibodies to these as well as other cell surface antigens are known and available in the art. By way of example, known antibody sequences that bind ASPH include, without limitation, FB50, a mouse monoclonal antibody that binds to the membrane proximal domain of ASPH; 622, a fully human antibody targeting the catalytic domain of ASPH; and 15c7, a murine mAb that binds the catalytic domain of ASPH, any of which can be utilized in development of a CE-BNP as disclosed. PSMA antibodies as disclosed in US Patent Application Publication 2023/0131727 to Goldberg et al., which is incorporated herein by reference thereto, can be utilized in some embodiments. In some embodiments, antibodies or fragments thereof as may be incorporated as targeting polypeptides can include IgG isotype monoclonal antibodies including, without limitation to, chimeric IgG1, human or humanized IgG1κ, and humanized IgG4κ, which have been utilized in previously described ADCs.

In addition to one or more exogenous targeting polypeptides, the engineered phage described herein can include one or more cancer therapies that are capable of killing or inhibiting the function of the cancer cell upon binding thereto. In order to ensure high delivery dose of the cancer therapies, a single engineered phage can generally include multiple, e.g., about 5 or more, about 10 or more, about 50 or more, or about 100 or more copies of a singly cancer therapy type. As discussed previously, the upper limit for the number of copies can vary, depending upon the particular phage type and coat protein(s) selected to form the fusion coat protein that carries the cancer therapy.

A cancer therapy delivered to a cancer cell by use of the CE-BNPs can be either proteinaceous or non-proteinaceous. When considering proteinaceous cancer therapies, the polypeptide can be directly or indirectly fused to the C-terminus, the N-terminus, or both terminals of a coat protein. In some embodiments, the proteinaceous cancer therapy can be fused to a polypeptide linker, which can in turn be fused (directly or indirectly) to a terminus of a coat protein. A cleavable linker sequence between the coat protein and the cancer therapy can be utilized in some embodiments, as it can allow for more efficient cleavage of the therapy from the phage subsequent to endocytosis by the cancer cell.

When considering non-proteinaceous cancer therapies, the engineered phage can include an exogenous polypeptide linker that can link the non-proteinaceous cancer therapy to the phage via the fusion coat protein that includes the exogenous polypeptide linker. As with proteinaceous cancer therapies, the presence of a cleavable linker can be utilized in some embodiments, so as to allow for efficient cleavage of the therapy from the phage subsequence to endocytosis by the cancer cell.

A proteinaceous cancer therapy can include a proteinaceous toxin that is toxic to the cancer cell. For example, toxins or fragments thereof as have been utilized in previously known immunotoxins can be incorporated in an engineered bacteriophage. A toxin of a CE-BNP may achieve the cytotoxic effect by mechanisms including tubulin binding, DNA binding, or topoisomerase inhibition. Bacterial toxins, plant toxins and fragments thereof including but not limited to pseudomonas exotoxin (e.g., exotoxin A from Pseudomonas aeruginosa, diphtheria toxin, shiga toxin, ricin (e.g., ricin A chain), saporins, dianthins, gelonin, abrin A, modeccin A chain, α-sacrin, certain Aleurites fordii proteins, certain Dianthin proteins, Phytolacca americana proteins (e.g., PAP, PAPII and PAP-S), Morodica charantia inhibitor, curcin, crotin, Saponaria officinalis inhibitor, gelonin, mitogillin, restrictocin, phenomycin, and enomycinetc, any of which or combination thereof may be used as deliverable toxic agents to kill cancer cells.

In some embodiments, the sequence of the toxin or toxin fragment may be modified such that potential human B and/or T-cell epitopes have been mutated to reduce potential immunogenicity as is known to one skilled in the art.

Exemplary toxin fragments as may be incorporated in an engineered phage can include, without limitation, PE-38 or PE38KDEL, which are fragments of pseudomonas exotoxin, the DT, which is a fragment of diphtheria toxin. PE-38 has been successfully employed in the FDA-approved recombinant immunotoxin, moxetumomab pasudotox-tdfk (Lumoxiti®), and DT has been used as part of the FDA-approved cytotoxins, denileukin diftitox (Ontak®) and tagraxofusp-erzs (Elzonris®).

In one embodiment, a CE-BNP as disclosed herein can incorporate a proteinaceous or non-proteinaceous inhibitor as a cancer therapy. For example, an engineered bacteriophage can include an inhibitor of lactic dehydrogenase (LDH). LDH is a key enzyme controlling the switch to aerobic glycolysis, which is often upregulated in cancer cells and is necessary to convert pyruvate to lactic acid and thus maintain the NAD⁺ levels of the cancer cells. Multiple isoforms of LDH are known to be expressed, with tumor cells relying primarily on LDHA. Small molecule inhibitors of LDHA have been explored as anti-cancer agents but lack specificity to the tumor cells. Through inclusion of such small molecule inhibitors in disclosed phage, which can combine the inhibitors with cancer cell targeting polypeptides, off-target effects of the therapy can be avoided. Peptide inhibitors of LDHA have also been identified that can be incorporated in disclosed phage and can inhibit activity by, e.g., disrupting protein-protein interactions that hold the active tetrameric complex together.

Examples of LDH inhibitors as may be incorporated in an engineered phage as disclosed herein can include, without limitation, FX11 (CAS 213971-34-7), gossypol (a nonselective inhibitor of LDH that blocks the binding of NADH, (Doherty et al., J. Clin. Invest., 2013, 123(9): 3685-3692), certain derivatives of 3-((3-carbamoyl-7-(3,5-dimethylisoxazol-4-yl)-6-methoxyquinolin-4-yl) amino) benzoic acid (Billiard et al., Cancer and Metabolism, 2013, 1(19): 1-17), LDH inhibitors as disclosed in U.S. Patent Application Publication No. 2020/0407397 to Mollapour et al., and U.S. Patent Application Publication No. 2020/0165233 to Klaveness et al., both of which being incorporated herein by reference thereto.

Cancer therapy as may be incorporated on an CE-BNP can include a drug, which can include a proteinaceous or non-proteinaceous drug (e.g., a small molecule cancer drug). A drug can be conjugated to the CE-BNP in such a way that it will not be released prior to internalization into the target cell, but can be released intracellularly to perform its function. In general, this can be accomplished by utilizing an exogenous polypeptide that includes an intracellular cleavable polypeptide linker to bind the drug to the phage.

There is no particular limit to the drug that can be conjugated to the CE-BNP. Anti-cancer agent including, but not limited to, cytotoxic agents, cytostatic agents, anti-angiogenic agents, debulking agents, chemotherapeutic agents, radiotherapeutic agents, targeted anti-cancer agents, biological response modifiers, cancer vaccines, cytokines, hormone therapies, oligonucleotides, antisense nucleotides, siRNAs, and anti-metastatic agents are encompassed herein.

Examples of cancer drugs encompassed herein can include, without limitation, inhibitors of tubulin polymerization such as maytansinoids (e.g., DM1) (see, e.g., U.S. Pat. Nos. 5,208,020, 5,416,06), auristatins (e.g., monmethyl auristatin DE (MMAE), monmethyle auristatin DF (MMAF)) (see, e.g., U.S. Pat. Nos. 5,635,483; 5,780,588; and 7,498,298), dolastatins (e.g., (DUO)40) and cryptophycins; topoisomerase inhibitors such as camptothecin derivatives like exactecan and deruxtecan (see, e.g., U.S. Pat. No. 6,630,579) or a camptothecin (CPT) analogue such as topotecan and irinotecan; DNA alkylating agents like duocarnycin or doxorubicin and derivatives thereof; enediyne antibiotics like esperamicin and calicheamicin or derivative thereof (see, e.g., U.S. Pat. Nos. 5,712,374; 5,714,586; 5,739,116; 5,767,285; 5,770,701; 5,770,710; 5,773,001; and 5,877,296), methotrexate; vindesine; a taxane such as docetaxel, paclitaxel; larotaxel; tesetaxel; ortataxel; a pyrrolobenzodiazepine (PBD) derivative (see, e.g., U.S. Pat. No. 10,639,373), an amatoxin derivative such as, α-amanitin and β-amanitin; and DNA minor groove binders such as pyrolobenodiazepine.

Drugs present on currently known antibody-drug conjugates (ADCs) can be incorporated on CE-BNP as disclosed. ADC approved by the FDA to treat cancer include gemtuzumab ozogamicin (Mylotarg™), brentuximab vedotin (Adcetris®), trastuzumab emtansine (Kadcyla®), inotuzumab ozogamicin (Besponsa®), polatuzumab vedotin-piiq (Polivy®), Enfortumab vedotin (Padcev®), and Trastuzumab deruxtecan (Enhertu®). One or more of the drugs included in these ADCs are encompassed herein.

In one embodiment, a CE-BNP can include a chelator that is capable of chelating radionuclides. A chelator can generally include a macrocyclic chelating moiety. Examples of macrocyclic chelating moieties include, without limitation, 1,4,7,10-tetraazacyclododecane-1,4,7,10,tetraacetic acid (DOTA), S-2-(4-isothiocyanatobenzyl)-1,4,7-triazacyclononane-1,4,7-triacetic acid (NOTA), 1,4,8,11-tetraazacyclodocedan-1,4,8,11-tetraacetic acid (TETA), 3,6,9,15-tetraazabicyclo[9.3.1]-pentadeca-1(15),11,13-triene-4-(S)-(4-isothiocyanatobenzyl)-3,6,9-triacetic acid (PCTA), 5-S-(4-aminobenzyl)-1-oxa-4,7,10-triazacyclododecane-4,7,10-tris(acetic acid) (DO3A), or a derivative thereof. Additional examples of chelators suitable for use in accordance with the present invention are described in U.S. Pat. Nos. 11,554,182 and 11,279,698, which are incorporated by reference herein. In those embodiments in which the CE-BNP includes multiple different chelators, any combination of chelators can be utilized.

In some embodiments, a radioactive metal ion can be bound to or coordinated to a chelator via coordinate bonding. In general, heteroatoms of a macrocyclic ring can participate in coordinate bonding of a metal ion to a chelator. Optionally, a chelator can be substituted with one or more substituent groups, and the one or more substituent groups can participate in coordinate bonding of a radiometal ion to a chelator in addition to, or alternatively to the heteroatoms of the macrocyclic ring.

Exemplary radioactive isotopes as may be incorporated in a CE-BNP can include γ-emitting, Auger-emitting, β-emitting, α-emitting or positron-emitting radioactive isotopes. Exemplary radioactive isotopes include. ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ¹³⁴Ce, ²²⁵Ac, and ²²⁷Ac, or any combination thereof.

In some embodiments, the radiometal ion can be a “therapeutic emitter,” meaning a radiometal ion that is useful in therapeutic applications such as to damage cancer cells. A suitable radiometal for use as a therapeutic agent is one that is capable of reducing or inhibiting the growth of or killing, a cancer cell. High energy radiometal ions as may be selected to a cancer cell can generally act over a short range so that the cytotoxic effects are localized to the targeted cells. Examples of therapeutic beta or alpha emitters include, but are not limited to, ¹³²La, ¹³⁵La, ¹³⁴Ce, ¹⁴⁴Nd, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, 186 Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²²⁵Ac, ²⁵⁵Fm, ²²⁷Th, ²²⁶Th, ²³⁰U, or any combination thereof.

Each of these radionuclides has its own special properties making it advantageous for radioimmunotherapy as is known to one skilled in the art. In the disclosed engineered phage, because of the number of potential sites available per CE-BNP, multiple radionuclides, for example one β and one α-emitter can be chelated to the same phage.

A polypeptide linker may be included on the CE-BNP as an extension of a phage coat protein in a fusion coat protein. In some embodiments, a linker can be designed to be readily reacted with a non-proteinaceous cancer therapy. This can be beneficial in some embodiments, as, because the targeting polypeptide is attached to the phage at separate sites, there can be little or no interference of the cancer therapy on the targeting proteins binding affinity. A polypeptide linker can generally be, but need not be, chemically stable to conditions outside the targeted cancer cell, and may be designed to cleave, immolate and/or otherwise specifically degrade inside the cancer cell. Alternatively, linkers that are not designed to specifically cleave or degrade inside the cancer cell may be used. Choice of externally stable versus unstable linkers may depend upon the toxicity of the cancer therapy. For cancer therapies that are toxic to normal cells, stable linkers can be preferred. For cancer therapies that are selective or targeted and have lower toxicity to normal cells stability in the extracellular environment can be less important. A wide variety of linkers useful for linking drugs to antibodies in the context of ADCs are known in the art and any of these peptidic linkers, as well as other linkers, may be used to link the cancer therapy to the phage of the disclosure.

The length of a polypeptide linker is not critical. For example a polypeptide linker can be from about 5 to about 50 amino acids long, such as from about 10 to about 40 amino acids long, such as from about 10 to about 35 amino acids long, such as from about 10 to about 30 amino acids long, such as from about 10 to about 25 amino acids long, such as from about 10 to about 20 amino acids long, such as from about 15 to about 20 amino acids long, in some embodiments.

Amino acid residues of a polypeptide linker component can include those occurring naturally as well as minor amino acids and non-naturally occurring amino acid analogs, such as citrulline. Exemplary amino acids that may be included in a linker are Gly, Ser Pro, Thr, Glu, Lys, Arg, lie, Leu, His and The. Exemplary linkers that may be used include Gly rich linkers, Gly and Ser containing linkers, Gly and Ala containing linkers, Ala and Ser containing linkers, and other flexible linkers. In some embodiments, a polypeptide linker can include a dipeptide, a tripeptide, a tetrapeptide or a pentapeptide. Exemplary dipeptides include valine-citrulline (vc or val-cit), alanine-phenylalanine (af or alaphe). Exemplary tripeptides include: glycine-valine-citrulline (gly-val-cit) and glycine-glycine-glycine (gly-gly-gly). In one embodiment, a polypeptide linker can include portions of an immunoglobulin hinge area, CL or CH1 derived from any immunoglobulin heavy or light chain isotype. Exemplary polypeptides as may be incorporated in an engineered phage as described herein include those described in US Patent Application Publication No. 2023/0125881 to Grinstaff et al., and 2023/0126689 to Schnabel et al.

In one embodiment, a linker can be a cleavable linker that is designed to largely release the cancer therapy once it has been delivered to the cancer cell, e.g., following endocytosis of the phage, which can substantially reduce undesirable non-specific toxicity by minimizing exposure of non-targeted cells and tissue to the cancer therapy, thereby providing an enhanced therapeutic benefit. Such a linker can include a cleavable linkage such as a disulfide linkage or a protease cleavage site. In one embodiment, a polypeptide linker can be designed and optimized in the selectivity for enzymatic cleavage by a particular enzymes, for example, a tumor-associated protease, extracellular metalloproteases, lysosomal proteases such as the cathepsins (cathepsin B, C and D), a plasmin protease, exo- and endo-peptidases, HIV proteases, as well as secretases, transferases, hydrolases, isomerases, ligases, oxidoreductases, esterases, glycosidases, phospholipases, endonucleases, furin, ribonucleases and β-lactamases.

Due to the large number of coat proteins of a phage, all of which can be substituted for fusion coat proteins in forming disclosed CE-BNP, an engineered phage can be designed to include additional beneficial materials at the surface, in addition to the targeting polypeptides and cancer therapies.

In one embodiment, a CE-BNP can include at the surface via a fusion coat protein a material that can decrease the chances of an immunogenic response by a subject. By way of example, a CE-BNP can include an IL-10 protein on the surface of the phage. IL-10 is known to suppress antigen presentation by professional antigen presenting cells (APCs) including dendritic cells and macrophages. By supplying IL-10 on the phage, rather than systemically, only APCs that are likely to endocytose and present phage derived proteins can be suppressed rather than all APC function. To enhance release of immunotoxins in target cells, subsequent to endocytosis, specific cleavable linkers may be employed such as those discussed above.

In some embodiments, an imaging agent or other detectable marked can be included on a CE-BNP. For example, a fluorescent protein such as green fluorescent protein (GFP) can be expressed on a phage as an extension of a coat protein for use as a marker.

In some embodiments a radioactive metal can be incorporated on a CE-BNP as an imaging agent or detectable label. Radionuclides used to radiolabel a phage can include, but are not limited to, ¹¹C, ¹³N, ¹⁵O, ¹⁸F, ⁶⁴Cu, ⁶⁷Cu, ⁶⁷Ga, ⁶⁸Ga, ^{81m}Kr, ⁸²Ru, ⁹⁹Tc, ¹¹¹Ir, ¹²³I, ¹²⁴I, ¹²⁵I, ¹³¹I, ¹³³Xe, ²⁰¹Th, ⁸⁹Zr, ⁹⁰Y, ¹⁷⁷Lu, ²¹¹At, ²¹²Pb, ²¹²Bi, ²¹³Bi, ¹³⁴Ce, and ²²⁵Ac, or any combination thereof.

In one embodiment, and in order to promote proper folding and disulfide bond formation of proteinaceous components of scFv, modified coat proteins of the engineered phage can co-express the yeast erv1p protein (FAD-linked sulfhydryl oxidase) and mammalian protein disulfide isomerase (PDI), which together have been demonstrated to afford mammalian-like disulfide bond formation in proteins expressed in bacteria. The versatility of this design makes it amenable to the incorporation of a large number of binding proteins onto a single phage while retaining the high copy number of each displayed protein.

In forming the engineered phage, a bacterial production system can be utilized in which the bacteria of choice can produce the phage that include multiple fusion coat proteins. In some embodiments, the coat protein of the starting phage may be silenced such that all coat protein of the product phage are fusion coat proteins. Alternatively, a portion of the native coat protein of the starting phage may be maintained to allow better separation/distribution of the fusion coat protein copies on the phage surface.

The engineered phage can be manufactured in bacterial cultures that can be grown at large scale in standard bio-fermenters. Expression plasmids, produced by recombinant DNA technology and encoding a phage fusion coat protein with an added extension encoding the targeting polypeptide, and also encoding a phage fusion coat protein with an added extension encoding a polypeptide cancer therapy and/or a polypeptide linker are transfected into the bacterial cells prior to infection with phage. By supplying multiple plasmids encoding different protein extensions added to the phage coat sequence or multiple copies of the gene encoding the phage coat protein with different added extensions encoding varying proteins of interest on the same plasmid, phage can be produced with the different proteins displayed on their coat.

Expression of the fusion coat proteins is achieved through stable transfection of bacteria with expression plasmids containing the sequences for the altered coat proteins prior to infection of the same bacteria with the starting phage. Specifically, a DNA sequence encoding the phage coat protein is ligated to a DNA sequence encoding the targeting polypeptide such that the latter sequence is in frame with the former. DNA encoding a short linker sequence may be placed between the two sequences if desired. In addition, a DNA sequence encoding the same or a different phage coat protein is ligated to a DNA sequence encoding the cancer therapy or a linker designed to be capable of conjugation to the non-proteinaceous cancer therapy such that the two are in frame with one another. In those embodiments in which the DNA sequence encodes the cancer therapy, a linker sequence that encodes the polypeptide linker may be placed between the two sequences.

The hybrid nucleic acid (e.g., DNA) sequences are each placed into one or more bacterial expression plasmid under the control of a bacterial expression promoter. A single bacterial expression plasmid can be utilized for both hybrid DNA sequences or each hybrid DNA sequence can be placed into a separate bacterial expression plasmid. In an embodiment in which a CE-BNP is formed that includes multiple targeting polypeptides and/or multiple cancer therapies, any combination thereof can be utilized, e.g., one or more bacterial expression plasmids that contain a single hybrid DNA sequence as well as one or more bacterial expression plasmids that contain multiple different hybrid DNA sequences. Moreover, variant copies of a hybrid DNA sequence encoding the different protein extensions desired may be expressed within the same or different expression plasmids. Transient expression systems have been used as tools of recombinant technology for many years and as such is not described in detail herein. By way of example and without limitation, suitable transient expression systems can include the pETDuet™ family of vectors from Novagen/EMD Millipore.

Promoters used for the expression plasmid(s) can be the same or different in different expression plasmids. Promoters can be an inducible promoter, a copy of the native phage promoter or any promoter deemed appropriate by one skilled in the art. When using different plasmids, it is generally recommended to use different selection markers to ensure that selected bacteria have incorporated all plasmid types. It is also possible for the different plasmids to use different strength promoters, thus allowing for the coat proteins with different extensions to be produced at varying levels which should allow for incorporation into the phage at different ratios. The plasmids are transfected into host bacterial cells that are infectable by the chosen phage. Host bacteria harboring the expression plasmids are subsequently infected with the starting phage and grown until lysis of the bacteria. If an inducible promoter is used in the expression plasmids, the inducing agent must be supplied during phage infection. Once bacterial cell lysis has occurred the product engineered phage can be purified and characterized using standard techniques. It should be noted that loss of infectivity by the modified phage is not a problem for the use of these CE-BNPs and in fact may be considered advantageous.

By way of example, when forming an engineered λ phage, an expression plasmid can include DNA encoding one or more fusion coat proteins based on one or more of the gpD, gpE or gpC coat proteins in conjunction with the encoding of one or more exogenous polypeptides in any combination. For example DNA of one or more plasmids can encode a targeting polypeptide in conjunction with a gpD coat protein, as well as a cancer therapy and/or a polypeptide linker in conjunction with a gpD coat protein. In another embodiment, DNA of one or more plasmids can encode a targeting polypeptide in conjunction with a gpD coat protein as well as a cancer therapy and/or a polypeptide linker in conjunction with a different, e.g., gpE, coat protein, or any combination thereof.

If an engineered M13 phage is to be formed, one or more of the pVIII, pIII, pVI, pVII or pIX proteins can generally be encoded in an expression plasmid in conjunction the targeting and cancer therapy related exogenous polypeptides. Similarly, the gp23 and/or gp24 proteins can generally be encoded when forming an engineered T4 bacteriophage and the gp10A and/or gp10B proteins can be encoded in an expression plasmid when forming an engineered T7 phage. For phage φX174 the gpF and/or gpG proteins can generally be encoded in conjunction with the exogenous polypeptides.

When using different expression plasmids to carry different fusion coat protein DNA, the regulatory components of the expression plasmids can be the same or differ from one another. For instance, in one embodiment, different expression plasmids can be essentially the same as one another, other than the fusion coat protein DNA sequences. In one embodiment, different selection markers can be incorporated on the different expression plasmids, which can be used to ensure that selected production bacteria have incorporated all plasmid types. In one embodiment, different plasmids or different expression components of a single plasmid can incorporate different promotors driving expression of the fusion coat proteins, for instance, different strength promoters, thus allowing for the fusion coat proteins with different exogenous polypeptide extensions to be produced at varying levels which can also allow for incorporation of the different fusion coat proteins into an engineered phage at different ratios.

The host bacterial cell can be any suitable type that can be transfected by the plasmid(s) and is also infectable by the phage that is to be the basis for the engineered phage product. For instance, when forming an engineered bacteriophage λ, the host bacterial cell can be an E. coli and an E. coli can thus be transfected with the expression plasmid(s) according to standard transfection practice. Suitable bacterial hosts for phage infection are known to those in the art. Depending upon the transfection/expression system utilized, additional components as necessary can be supplied to the bacterial host. For instance, if an inducible promoter is incorporated in the expression plasmid(s), the inducing agent can also be supplied to the bacterial host during phage infection.

Upon transfection and infection, the bacterial host can produce the engineered bacteriophage that incorporate the fusion coat proteins. Beneficially, because the fusion coat proteins are produced from plasmid(s) transiently expressed in the bacteria during phage production, the DNA encoding the exogenous polypeptide is not incorporated into the phage.

The amount of fusion coat proteins incorporated into an engineered bacteriophage can be controlled in one embodiment, such as through selection of the promoter strength of an expression plasmid. Such an approach can be used to control relative amount of different fusion coat protein in a bacteriophage as well as relative amount of the starting, e.g., wild type, coat protein vs. the fusion coat protein. In such an embodiment, the phage coat protein upon which the fusion coat protein is based can be maintained to a controlled extent on the engineered phage. Thus, the engineered phage can include a portion of the coat protein lacking any fused exogenous polypeptide in addition to the fused coat protein.

In one embodiment, the bacterial cell can be infected with a knock-out phage in which the wild-type coat protein expression has been silenced or deleted. In this case, all of the coat protein of the type incorporated in the expression plasmid (e.g., all gpD coat protein of a bacteriophage λ) can be present in the expressed engineered phage as fusion coat protein.

In one embodiment, the bacterial cell can be infected with a phage that has been genetically altered as compared to a wild-type phage. For instance, the starting phage that infect the bacteria can be modified to include a stop codon that prevents formation of a coat protein. In such a case, the bacterial hose can include a genetic modification to avoid that stop codon, but the product engineered phage can still include the stop codon and as such, will be less infective during use. In another embodiment, the phage can be genetically altered as compared to a wild-type phage so as to be less immunogenic in the intended use, e.g., to a subject to be treated by use of the CE-BNP.

Following lysis, engineered bacteriophage may be purified by any number of methods known to those skilled in the art for bacteriophage purification. These methods include, but are not limited to polyethylene glycol (PEG) precipitation, tangential flow filtration, affinity chromatography, etc. By way of example, phage can be isolated via a series of steps involving centrifugation of lysed cultures, tangential flow filtration for concentration and buffer exchange, ethanol and triton X-114 precipitation to remove endotoxin followed by additional filtration, concentration and washing of the material. Engineered bacteriophage characterized by standard methods known to those skilled in the art. Phage yields can be on the order of 5×10¹³-5×10¹⁴ particles/liter.

Product characterization methods can include, without limitation:
Purity: pH; potentiometric determination; visual appearance, sterility; spectrophotometry and ELISA to determine level of chemical impurities. The goal is to achieve >95% purity.
Microbial contaminants: Limulus Amebocyte Lysate test to determine level of endotoxins.
Particle count and size: Particle numbers and size are determined by NTA using a NS300 instrument (Malvern Panalytical) equipped with a blue laser (488 nm). Phage size is ˜70-100 nm.
Identity: SDS-PAGE, Dot and Western blotting with specific antibodies to detect antigen presence on phage particles.
Determination of numbers of displayed proteins per phage: An important aspect of characterization of the CE-BNP will be to determine the relative numbers of each protein displayed per phage. To accomplish this, we will develop a novel method employing mass spectrometry-based parallel reaction monitoring (PRM) applying the recent methodology of Lavado-Garcia, et al. 50. Briefly, concentrations of phage as determined by NTA are subjected to trypsin digestion. Target peptides of interest are selected based on the locations of known variant mutations, and corresponding peptides are synthesized to incorporate 13C or 15N for rapid identification and quantitation. Synthesized peptides are added to tryptic digestions and purified by reverse phase HPLC on a C18 column. Resulting peptide mixtures are analyzed by LC/MS-MS. Numbers of resulting peptides for specific antigens can be divided by original particle numbers determined by NTA to calculate the number of antigens per phage. A control peptide from gpD and/or gpE will be used to obtain the total number of gpD/gpE and modified gpD/gpE proteins per phage.

Subsequent to construction, purification and characterization of CE-BNPs, they may be formulated for human administration using standard protocols well known to those skilled in the art. Generally, bacteriophage are highly soluble in saline solutions. Routes of administration may include, but are not limited to, intravenous, intramuscular, intraperitoneal and/or intradermal.

A therapeutic composition including the CE-BNP can be formed according to protocols as are known to those skilled in the art. For instance, purified engineered bacteriophage can be transferred into a buffered saline solution with commonly used preservatives and filter sterilized. Because of the high stability of bacteriophage, a therapeutic composition incorporating an engineered bacteriophage can be stable at ambient and room temperatures for long periods, e.g., one week to several months.

A therapeutic composition can be prepared in one embodiment as an injectable, either as a liquid solution or suspension. A solid form suitable for solution in, or suspension in, liquid prior to injection may also be prepared. The preparation may also be emulsified, or the ingredients can be mixed with excipients that are pharmaceutically acceptable and compatible with the bacteriophage. Suitable excipients are, for example, saline or buffered saline (pH 7 to 8), or other physiologic, isotonic solutions that may also contain dextrose, glycerol or the like and combinations thereof. In addition, a therapeutic composition can contain minor amounts of auxiliary substances such as wetting or emulsifying agents, pH buffering agents that can enhance the effectiveness of the vaccine.

A therapeutic composition can be prepared in one embodiment as an inhalable composition. For instance, an inhalable therapeutic composition can include the engineered bacteriophage as individual particles or as a component of a larger particle or droplet in which the particle/droplet size facilitates penetration throughout the lungs. In one embodiment, a therapeutic composition designed to be inhaled from a dry powder inhaler can include dry particles comprising engineered bacteriophage as described. In one embodiment, an inhalable composition can include particles or droplets comprising engineered bacteriophage suspended in a propellant, e.g., in the form of an aerosol. In one embodiment, an inhalable composition can be a suspension of droplets or particles comprising engineered bacteriophage held in a liquid carrier that can be intended for administration by use of a liquid nebulizer system. In such an embodiment, a therapeutic composition can incorporate an aqueous liquid carrier, a nonaqueous liquid carrier, or can include a combination of an aqueous and nonaqueous carrier.

A pharmaceutical composition can include individual particles or droplets having a size that can permit penetration into the alveoli of the lungs, generally about 10 μm or less in size, about 7.5 μm or less in size, or about 5 μm or less in size in some embodiments. For instance, when considering aerodynamically light particles (e.g., having a bulk density of about 0.5 g/cm3 or less) for delivery as a dry powder formulation, a pharmaceutical composition can carry larger particles, for instance having a size of from about 5 μm to about 30 μm.

A therapeutic composition can be delivered by any of the standard routes including but not limited to intramuscular, intravenous, subcutaneous, intradermal, inhalation, etc. A therapeutic composition can be applied or instilled into body cavities, absorbed through the skin (e.g., via a transdermal patch), inhaled, ingested, topically applied to tissue, or administered parenterally via, for instance, intravenous, peritoneal, or intraarterial administration.

A delivery device can be utilized that allows controlled or sustained release, such as a sponge, biocompatible meshwork, mechanical reservoir, or mechanical implant. Implants and devices as can be useful for administration of a therapeutic composition have been described and are known in the art (see, e.g., U.S. Pat. Nos. 5,443,505 and 4,863,457, both of which are incorporated by reference herein). A therapeutic composition can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

An engineered phage can be used to control and/or treat existing disease and or prophylactically to prevent disease when an individual is concerned about being exposed to a pathogen.

The dosage of a therapeutic composition administered to a subject can depend on a number of factors, including the extent of any side-effects, the particular route of administration, and the like. The dose ideally comprises an “effective amount” of a therapeutic composition, i.e., a dose of the binding agent polypeptide carried on an engineered bacteriophage that can prevent activity of the pathogen of interest or otherwise interfere with the disease process.

The present invention may be better understood with reference to the Examples set forth below.

### Example 1

Expression of anti-ASPH targeting protein and a toxin on the surface of a lambda phage was carried out.

An expression plasmid encoding a sequence to gpD phage coat protein in frame with a sequence to an scFv against ASPH, FB50, was transfected into E. coli bacteria. FB50 is derived from mouse monoclonal antibody and exhibits high affinity to the membrane proximal extracellular domain. A second expression plasmid encoding a sequence to gpD phage coat protein in frame with a sequence to express Pseudomonas exotoxin (PE38) was also transfected into the bacteria. PE38 is the toxin used in the FDA approved immunotoxin Lumoxiti® of AstraZeneca. The bacteria was subsequently infected with phage λ.

The phage produced from the bacteria was collected and isolated. The presence of the anti-ASPH targeting protein was verified by demonstrating binding of the isolated phage to a recombinant ASPH protein. The presence of the PE38 toxin was verified by reactivity to anti-PE38 antibody.

### Example 2

Expression of anti-ASPH targeting protein and a toxin on the surface of a lambda phage was carried out.

An expression plasmid encoding a sequence to gpD phage coat protein in frame with a sequence to an scFv against ASPH, 15C7, was transfected into E. coli bacteria. 15C7 is derived from mouse monoclonal antibody and exhibits high affinity to the catalytic domain. A second expression plasmid encoding a sequence to gpD phage coat protein in frame with a sequence to express PE38 was also transfected into the bacteria. The bacteria was subsequently infected with phage λ.

The phage produced from the bacteria was collected and isolated. The presence of the anti-ASPH targeting protein was verified by demonstrating binding of the isolated phage to a recombinant ASPH protein. The presence of the PE38 toxin was verified by reactivity to anti-PE38 antibody.

### Example 3

Expression of anti-ASPH targeting protein and a toxin on the surface of a lambda phage was carried out.

An expression plasmid encoding a sequence to gpD phage coat protein in frame with a sequence to an scFv against ASPH, 622, was transfected into E. coli bacteria. 622 is a fully human sequence and exhibits moderate affinity to the catalytic domain. A second expression plasmid encoding a sequence to gpD phage coat protein in frame with a sequence to express PE38 was also transfected into the bacteria. The bacteria was subsequently infected with phage λ.

The phage produced from the bacteria was collected and isolated. The presence of the anti-ASPH targeting protein was verified by demonstrating binding of the isolated phage to a recombinant ASPH protein. The presence of the PE38 toxin was verified by reactivity to anti-PE38 antibody.

### Example 4

Two different CE-BNP were formed and compared for efficacy against a pancreatic cancer cell line and against a prostate cancer cell line. Both cancer cell lines express ASPH and the prostate cancer cell line also expresses PSMA

The first CE-BNP expressed the anti-ASPH scFv 15C7 and the PE38 toxin as described in Example 2, above.

The second CE-BNP expressed an anti-PSMA antibody and the PE38 toxin. An expression plasmid encoding a sequence to gpD phage coat protein in frame with a sequence to an scFv against PSMA was transfected into E. coli bacteria. A second expression plasmid encoding a sequence to gpD phage coat protein in frame with a sequence to express PE38 was also transfected into the bacteria. The proteins were expressed as C-terminal extensions of gpD phage coat protein within the bacteria prior to infection with phage λ. Bacteria were infected with phage λ. Lambda phage produced contained 3 forms of the gpD protein in roughly equal amounts; gpD, gpD-scFv, and gpD-PE38.

The two cancer cell lines were incubated with each of the CE-BNP.

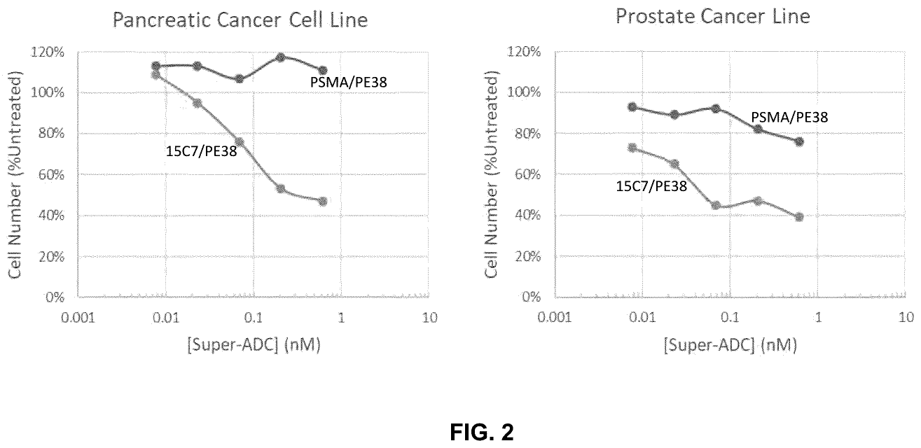

Results are shown in FIG. 2. As can be seen, the 15C7/PE38 CE-BNP was effective against both cell lines and the PSMA/PE38 was effective against the prostate cell line, which is to be expected at the pancreatic cell line does not express PSMA.

### Prophetic Example 1

Toxin loaded CE-BNP for the treatment of prostate cancer—one toxin and one target antigen, phage λ.

Lambda phage are used as the BNP. scFv against PSMA are used to specifically target prostate cells. Pseudomonas exotoxin (PE38) serves as the toxin. Both the scFv against PSMA as well as the pseudomonas exotoxin (PE38) are expressed as C-terminal extensions of gpD phage coat protein within the bacteria prior to infection with phage λ. A linker containing a furin cleavable protease site is placed between the gpD and PE38 in that construct. Lambda phage produced should contain 3 forms of the gpD protein in roughly equal amounts; gpD, gpD-scFv, and gpD-linker-PE38. These CE-BNP are used to treat prostate cancer.

### Prophetic Example 2

Multi-toxin loaded CE-BNP for the treatment of prostate cancer—two toxins and one target antigen, phage λ

Lambda phage are used as the BNP. scFv against PSMA are used to specifically target prostate cells. Pseudomonas exotoxin (PE38) and diphtheria toxin A chain (DT) serve as the toxins. All three proteins are expressed as c-terminal extensions of gpD phage coat protein within the bacteria prior to infection with phage λ. A linker containing a furin cleavable protease site is placed between the gpD and both the PE38 and the DT in that construct. Lambda phage produced should contain 4 forms of the gpD protein in roughly equal amounts; gpD, gpD-scFv, gpD-linker-PE38 and gpD-linker-DT. These CE-BNP are used to treat prostate cancer.

### Prophetic Example 3

Toxin loaded multivalent CE-BNP for the treatment of prostate cancer—one toxin and two target antigens, phage λ.

Lambda phage are used as the BNP. scFv against PSMA and scFv against ASPH are used to specifically target prostate cancer cells. Pseudomonas exotoxin (PE38) serves as the toxin. Both the scFv against PSMA and ASPH as well as the pseudomonas exotoxin (PE38) are expressed as c-terminal extensions of gpD phage coat protein within the bacteria prior to infection with phage λ. A linker containing a furin cleavable protease site is placed between the gpD and PE38 in that construct. Lambda phage produced should contain 3 forms of the gpD protein in roughly equal amounts; gpD, gpD-scFv(PSMA), gpD-scFv(ASPH) and gpD-linker-PE38. These CE-BNP are used to treat prostate cancer.

### Prophetic Example 4

Multi-toxin loaded multivalent CE-BNP for the treatment of prostate cancer—two toxins and two target antigens, phage λ.

Lambda phage are used as the BNP. scFv against PSMA and scFv against ASPH are used to specifically target prostate cancer cells. Pseudomonas exotoxin (PE38) and diphtheria toxin A chain (DT) serve as the toxins. All four proteins are expressed as c-terminal extensions of gpD phage coat protein within the bacteria prior to infection with phage λ. A linker containing a furin cleavable protease site is placed between the gpD and both the PE38 and the DT in that construct. Lambda phage produced should contain 5 forms of the gpD protein in roughly equal amounts; gpD, gpD-scFv(PSMA), gpD-scFv(ASPH), gpD-linker-PE38 and gpD-linker-DT. These CE-BNP are used to treat prostate cancer.

### Prophetic Example 5

Toxin loaded CE-BNP for the treatment of prostate cancer—one toxin and one target antigen, M13 phage.

M13 phage are used as the BNP. scFv against PSMA are used to specifically target prostate cells. Pseudomonas exotoxin (PE38) serves as the toxin. Both the scFv against PSMA as well as the pseudomonas exotoxin (PE38) are expressed as C-terminal extensions of gpD phage coat protein within the bacteria prior to infection with phage λ. A linker containing a furin cleavable protease site is placed between the gpD and PE38 in that construct. Lambda phage produced should contain 3 forms of the gpD protein in roughly equal amounts: gpD, gpD-scFv, and gpD-linker-PE38. These CE-BNP are used to treat prostate cancer.

### Prophetic Example 6

Toxin loaded CE-BNP for the treatment of multiple myeloma—one toxin and one target antigen, phage λ.

Lambda phage are used as the BNP. scFv against BCMA are used to specifically target multiple myeloma cells. Pseudomonas exotoxin (PE38) serves as the toxin. Both the scFv against BCMA as well as the pseudomonas exotoxin (PE38) are expressed as C-terminal extensions of gpD phage coat protein within the bacteria prior to infection with phage λ. A linker containing a furin cleavable protease site is placed between the gpD and PE38 in that construct. Lambda phage produced should contain 3 forms of the gpD protein in roughly equal amounts: gpD, gpD-scFv, and gpD-linker-PE38. These CE-BNP are used to treat multiple myeloma.

### Prophetic Example 7

Drug conjugated CE-BNP for the treatment of prostate cancer—one drug and one target antigen, phage λ.

Lambda phage are used as the BNP. scFv against PSMA are used to specifically target prostate cells. A short peptide containing a linker and a cathepsin B protease site and a cysteine residue will be expressed as a means of conjugating drug to the phage. Both the scFv against PSMA as well as peptide are expressed as C-terminal extensions of gpD phage coat protein within the bacteria prior to infection with phage λ. Lambda phage produced should contain 3 forms of the gpD protein in roughly equal amounts; gpD, gpD-scFv, and gpD-peptide. The drug, a maytansinoid derivative, will be conjugated to resulting phage. These CE-BNP are used to treat prostate cancer.

### Prophetic Example 8

Multi-drug conjugated CE-BNP for the treatment of prostate cancer—two drugs and one target antigen, phage λ.

Lambda phage are used as the BNP. scFv against PSMA are used to specifically target prostate cells. A short peptide containing a linker and a cathepsin B protease site and a cysteine residue will be expressed as a means of conjugating drug to the phage. Both the scFv against PSMA as well as peptide are expressed as C-terminal extensions of gpD phage coat protein within the bacteria prior to infection with phage λ. Lambda phage produced should contain 3 forms of the gpD protein in roughly equal amounts; gpD, gpD-scFv, and gpD-peptide. Two drugs, a maytansinoid derivative as well as an auristatin derivative, will be conjugated to resulting phage. These CE-BNP are used to treat prostate cancer.

### Prophetic Example 9

Drug conjugated multivalent CE-BNP for the treatment of prostate cancer—one drug and two target antigens, phage λ.

Lambda phage are used as the BNP. scFv against PSMA and scFv against ASPH are used to specifically target prostate cells. A short peptide containing a linker and a cathepsin B protease site and a cysteine residue will be expressed as a means of conjugating drug to the phage. The scFv against PSMA, the scFv against ASPH and the peptide are expressed as C-terminal extensions of gpD phage coat protein within the bacteria prior to infection with phage λ. Lambda phage produced should contain 4 forms of the gpD protein in roughly equal amounts; gpD, gpD-scFv(PSMA), gpD-scFv(ASPH), and gpD-peptide. The drug, a maytansinoid derivative, will be conjugated to resulting phage. These CE-BNP are used to treat prostate cancer.

### Prophetic Example 10

Multi-drug conjugated multivalent CE-BNP for the treatment of prostate cancer—two drugs and two target antigens, phage λ.

Lambda phage are used as the BNP. scFv against PSMA and scFv against ASPH are used to specifically target prostate cells. A short peptide containing a linker and a cathepsin B protease site and a cysteine residue will be expressed as a means of conjugating drug to the phage. The scFv against PSMA, the scFv against ASPH and the peptide are expressed as c-terminal extensions of gpD phage coat protein within the bacteria prior to infection with phage λ. Lambda phage produced should contain 4 forms of the gpD protein in roughly equal amounts; gpD, gpD-scFv(PSMA), gpD-scFv(ASPH), and gpD-peptide. Two drugs, a maytansinoid derivative as well as an auristatin derivative, will be conjugated to resulting phage. These CE-BNP are used to treat prostate cancer.

### Prophetic Example 11

Drug conjugated CE-BNP for the treatment of prostate cancer—one drug and one target antigen, M13 phage.

M13 phage are used as the BNP. scFv against PSMA are used to specifically target prostate cells. A short peptide containing a linker and a cathepsin B protease site and a cysteine residue will be expressed as a means of conjugating drug to the phage. Both the scFv against PSMA as well as peptide are expressed as c-terminal extensions of gpD phage coat protein within the bacteria prior to infection with phage λ. Lambda phage produced should contain 3 forms of the gpD protein in roughly equal amounts; gpD, gpD-scFv, and gpD-peptide. The drug, a maytansinoid derivative, will be conjugated to resulting phage. These CE-BNP are used to treat prostate cancer.

### Prophetic Example 12

Drug conjugated CE-BNP for the treatment of multiple myeloma—one drug and one target antigen, phage λ.

Lambda phage are used as the BNP. scFv against BCMA are used to specifically target multiple myeloma cells. A short peptide containing a linker and a cathepsin B protease site and a cysteine residue will be expressed as a means of conjugating drug to the phage. Both the scFv against PSMA as well as peptide are expressed as c-terminal extensions of gpD phage coat protein within the bacteria prior to infection with phage λ. Lambda phage produced should contain 3 forms of the gpD protein in roughly equal amounts; gpD, gpD-scFv, and gpD-peptide. The drug, a maytansinoid derivative, will be conjugated to resulting phage. These CE-BNP are used to treat multiple myeloma.

### Prophetic Example 13

Radionuclide chelated CE-BNP for the treatment of prostate cancer—one radionuclide and one target antigen, phage λ.

Lambda phage are used as the BNP. scFv against PSMA are used to specifically target prostate cells. A short peptide containing a linker and a cysteine residue will be expressed as a means of conjugating chelator to the phage. Both the scFv against PSMA as well as peptide are expressed as C-terminal extensions of gpD phage coat protein within the bacteria prior to infection with phage λ. Lambda phage produced should contain 3 forms of the gpD protein in roughly equal amounts; gpD, gpD-scFv, and gpD-peptide. The chelator, DOTA, will be conjugated to resulting phage. These CE-BNP are used to treat prostate cancer. Immediately prior to treatment use, the phage will be loaded with the α-emitter, ²¹³Bi.

### Prophetic Example 14

Multi-radionuclide chelated CE-BNP for the treatment of prostate cancer—two radionuclides and one target antigen, phage λ.

Lambda phage are used as the BNP. scFv against PSMA are used to specifically target prostate cells. A short peptide containing a linker and a cysteine residue will be expressed as a means of conjugating chelator to the phage. Both the scFv against PSMA as well as peptide are expressed as C-terminal extensions of gpD phage coat protein within the bacteria prior to infection with phage λ. Lambda phage produced should contain 3 forms of the gpD protein in roughly equal amounts; gpD, gpD-scFv, and gpD-peptide. The chelator, DOTA, will be conjugated to resulting phage. These CE-BNP are used to treat prostate cancer. Immediately prior to treatment use, the phage will be loaded with the α-emitter, ²¹³Bi, and the β-emitter, 90Y.

### Prophetic Example 15

Radionuclide chelated multivalent CE-BNP for the treatment of prostate cancer—one radionuclide and two target antigens, phage λ.

Lambda phage are used as the BNP. scFv against PSMA and scFv against ASPH are used to specifically target prostate cells. A short peptide containing a linker and a cysteine residue will be expressed as a means of conjugating chelator to the phage. Both the scFv against PSMA as well as peptide are expressed as C-terminal extensions of gpD phage coat protein within the bacteria prior to infection with phage λ. Lambda phage produced should contain 4 forms of the gpD protein in roughly equal amounts; gpD, gpD-scFv(PSMA), gpD-scFv(ASPH), and gpD-peptide. The chelator, DOTA, will be conjugated to resulting phage. These CE-BNP are used to treat prostate cancer. Immediately prior to treatment use, the phage will be loaded with the α-emitter, ²¹³Bi.

### Prophetic Example 16

Multi-radionuclide chelated multivalent CE-BNP for the treatment of prostate cancer—two radionuclides and two target antigens, phage λ.

Lambda phage are used as the BNP. scFv against PSMA and scFv against ASPH are used to specifically target prostate cells. A short peptide containing a linker and a cysteine residue will be expressed as a means of conjugating chelator to the phage. Both the scFv against PSMA as well as peptide are expressed as C-terminal extensions of gpD phage coat protein within the bacteria prior to infection with phage λ. Lambda phage produced should contain 4 forms of the gpD protein in roughly equal amounts; gpD, gpD-scFv(PSMA), gpD-scFv(ASPH), and gpD-peptide. The chelator, DOTA, will be conjugated to resulting phage. These CE-BNP are used to treat prostate cancer. Immediately prior to treatment use, the phage will be loaded with the α-emitter, ²¹³Bi, and the β-emitter, ⁹⁰Y.

### Prophetic Example 17

Radionuclide chelated CE-BNP for the treatment of prostate cancer—one radionuclide and one target antigen, M13 phage.

M13 phage are used as the BNP. scFv against PSMA are used to specifically target prostate cells. A short peptide containing a linker and a cysteine residue will be expressed as a means of conjugating chelator to the phage. Both the scFv against PSMA as well as peptide are expressed as c-terminal extensions of gpD phage coat protein within the bacteria prior to infection with phage λ. Lambda phage produced should contain 3 forms of the gpD protein in roughly equal amounts; gpD, gpD-scFv, and gpD-peptide. The chelator, DOTA, will be conjugated to resulting phage. These CE-BNP are used to treat prostate cancer. Immediately prior to treatment use, the phage will be loaded with the α-emitter, ²¹³Bi.

### Prophetic Example 18

Radionuclide chelated CE-BNP for the treatment of multiple myeloma—one radionuclide and one target antigen, phage λ.

Lambda phage are used as the BNP. scFv against BCMA are used to specifically target multiple myeloma cells. A short peptide containing a linker and a cysteine residue will be expressed as a means of conjugating chelator to the phage. Both the scFv against PSMA as well as peptide are expressed as C-terminal extensions of gpD phage coat protein within the bacteria prior to infection with phage λ. Lambda phage produced should contain 3 forms of the gpD protein in roughly equal amounts; gpD, gpD-scFv, and gpD-peptide. The chelator, DOTA, will be conjugated to resulting phage. These CE-BNP are used to treat multiple myeloma. Immediately prior to treatment use, the phage will be loaded with the α-emitter, ²¹³Bi.

### Prophetic Example 19

Toxin and drug loaded CE-BNP for the treatment of prostate cancer—one toxin, one drug and one target antigen, phage λ.

Lambda phage are used as the BNP. scFv against PSMA are used to specifically target prostate cells. Pseudomonas exotoxin (PE38) serves as the toxin and a maytansinoid derivative serves as the drug. A short peptide containing a linker and a cathepsin B protease site and a cysteine residue will be expressed as a means of conjugating drug to the phage. The two proteins and the peptide are expressed as C-terminal extensions of gpD phage coat protein within the bacteria prior to infection with phage λ. A linker containing a furin cleavable protease site is placed between the gpD and the PE38 in that construct. Lambda phage produced should contain 4 forms of the gpD protein in roughly equal amounts; gpD, gpD-scFv, gpD-linker-PE38 and gpD-linker-peptide. The drug, a maytansinoid derivative, will be conjugated to resulting phage. These CE-BNP are used to treat prostate cancer.

### Prophetic Example 20

Toxin and radionuclide loaded CE-BNP for the treatment of prostate cancer—one toxin, one radionuclide and one target antigen, phage λ.

Lambda phage are used as the BNP. scFv against PSMA are used to specifically target prostate cells. Pseudomonas exotoxin (PE38) serves as the toxin and a maytansinoid derivative serves as the drug. A short peptide containing a linker and a cysteine residue will be expressed as a means of conjugating a chelator to the phage. The proteins and the peptide are expressed as C-terminal extensions of gpD phage coat protein within the bacteria prior to infection with phage λ. A linker containing a furin cleavable protease site is placed between the gpD and the PE38 in that construct. Lambda phage produced should contain 4 forms of the gpD protein in roughly equal amounts; gpD, gpD-scFv, gpD-linker-PE38 and gpD-peptide. The chelator, DOTA, will be conjugated to resulting phage. These CE-BNP are used to treat multiple myeloma. Immediately prior to treatment use, the phage will be loaded with the α-emitter, ²¹³Bi.

### Prophetic Example 21

Drug and radionuclide loaded CE-BNP for the treatment of prostate cancer—one drug, one radionuclide and one target antigen, phage λ.

Lambda phage are used as the BNP. scFv against PSMA are used to specifically target prostate cells. A short peptide containing a linker and a cathepsin B protease site and a cysteine residue will be expressed as a means of conjugating drug to the phage. The protein and the peptide are expressed as C-terminal extensions of gpD phage coat protein within the bacteria prior to infection with phage λ. Lambda phage produced should contain 3 forms of the gpD protein in roughly equal amounts; gpD, gpD-scFv and gpD-linker-peptide. The drug, a maytansinoid derivative, and the chelator, DOTA will be conjugated to resulting phage. These CE-BNP are used to treat prostate cancer. Immediately prior to treatment use, the phage will be loaded with the α-emitter, ²¹³Bi.

### Prophetic Example 22

Toxin, drug and radionuclide loaded CE-BNP for the treatment of prostate cancer—one toxin, one drug and one target antigen, phage λ.

Lambda phage are used as the BNP. scFv against PSMA are used to specifically target prostate cells. Pseudomonas exotoxin (PE38) serves the toxin and a maytansinoid derivative serves as the drug. A short peptide containing a linker and a cathepsin B protease site and a cysteine residue will be expressed as a means of conjugating drug to the phage. The protein and the peptide are expressed as C-terminal extensions of gpD phage coat protein within the bacteria prior to infection with phage λ. A linker containing a furin cleavable protease site is placed between the gpD and the PE38 in that construct. Lambda phage produced should contain 4 forms of the gpD protein in roughly equal amounts; gpD, gpD-scFv, gpD-linker-PE38 and gpD-linker-peptide. The drug, a maytansinoid derivative, and the chelator, DOTA will be conjugated to resulting phage. These CE-BNP are used to treat prostate cancer. Immediately prior to treatment use, the phage will be loaded with the α-emitter, ²¹³Bi.

### Prophetic Example 23

gpD/gpE fusion proteins will be constructed with scFv targeted against ASPH and peptide inhibitors of LDHA tetramerization.

Sequences of three antibodies known to bind to ASPH; FB50, a murine mAb that binds to the membrane proximal domain, 15c7, a murine mAb that binds to the catalytic domain, and 622, a fully human Ab targeting the catalytic domain will be used as targeting polypeptides. Using the known variable heavy and light chain domain sequences of the antibodies, scFv will be engineered for expression as fusion proteins with phage λ gpD or gpE capsid proteins. Commercially available bacterial expression vectors (pET Duet series, Novagen) will be used. Four vectors are available with different antibiotic resistance allowing for simultaneous expression of up to 8 different proteins. These vectors will be used to express scFv as C-terminal extensions of gpD or gpE using a short linker of glycine and serine residues. The system will be designed for singular or simultaneous expression of all three scFv; derived from FB50, 15C7 and 622.

Inhibitory peptides that block LDHA trimerization including a series of five 6-7 amino acid peptides based on residues 5-17 of LDHA and a 16 amino acid peptide corresponding to residues 60-75 of LDHA will be used. These peptides will be expressed as C-terminal fusion proteins with gpD or gpE. A cleavable linker will be used between the protein and the peptide to allow for intracellular release of peptide. Linkers will be based on the cathepsin B (lysosomal) protease cleavage site and the furin (Golgi) protease cleavage site.

All plasmids will be verified via sequencing of the final constructs. Proteins will be expressed in bacteria and expression will be verified by SDS-PAGE. Functionality of the gpD (or gpE)/scFv fusion proteins will be checked by an ELISA based binding assay using biotinylated recombinant ASPH. LDH peptide fusion proteins will be cleaved in vitro using the appropriate protease and tested for inhibitory activity in a standard spectrophotometric LDH enzyme assay monitoring conversion of NADH to NAD+.

Phage displaying the fusion proteins will be produced in bacterial culture by coinfecting bacteria expressing the requisite fusion proteins with phage λ. Established protocols will be utilized for phage production, isolation and purification including endotoxin removal. Resulting phage will be characterized for a) binding to biotinylated recombinant ASPH and b) ability to release upon protease cleavage active inhibitory peptides as measured using the LDH assay.

### Prophetic Example 24

Demonstration of cellular killing and/or reduction of glycolytic rate in leukemia cell lines upon treatment with CE-BNP.

Human leukemic cell lines will be purchased from ATCC and maintained in culture. Binding of CE-BNP formed as described above to cells will be measured by flow cytometry using phage labeled with SYBR Gold while maintaining cells at 4° C. to avoid endocytosis. Endocytosis will be demonstrated by uptake of labeled phage into cells and visualization by fluorescent microscopy. Measure of glycolytic rate is performed by monitoring production of lactic acid and cell killing is determined by a standard viability (MTS) assay.

While certain embodiments of the disclosed subject matter have been described using specific terms, such description is for illustrative purposes only, and it is to be understood that changes and variations may be made without departing from the spirit or scope of the subject matter.

What is claimed is:

1. An engineered multivalent, multiplexed bacteriophage comprising:
a first fusion coat protein that includes a first exogenous polypeptide fused to a first bacteriophage coat protein, the first exogenous polypeptide comprising a first targeting polypeptide that includes a first binding sequence that binds a first cancer cell surface antigen;
a second fusion coat protein that includes a second exogenous polypeptide fused to a second bacteriophage coat protein, the second exogenous polypeptide comprising a first cancer therapy and/or comprising a polypeptide linking agent; wherein
the engineered multivalent, multiplexed bacteriophage includes multiple copies of the first fusion coat protein and multiple copies of the second fusion coat protein,
the engineered multivalent, multiplexed bacteriophage is free of nucleic acids that encode the first and second exogenous polypeptides, and
the engineered multivalent, multiplexed bacteriophage includes the bacteriophage genome.

2. The engineered multivalent, multiplexed bacteriophage of claim 1, wherein the second fusion coat protein comprises the first cancer therapy.

3. The engineered multivalent, multiplexed bacteriophage of claim 2, wherein the second fusion coat protein comprises the first cancer therapy and the polypeptide linking agent between the second coat protein and the first cancer therapy.

4. The engineered multivalent, multiplexed bacteriophage of claim 1, wherein the second fusion coat protein comprises the polypeptide linking agent, and wherein the first cancer therapy is a non-proteinaceous cancer therapy that is bonded to the polypeptide linking agent.

5. The engineered multivalent, multiplexed bacteriophage of claim 1, wherein the bacteriophage is selected from the group consisting of bacteriophage λ, bacteriophage M13, bacteriophage T4, bacteriophage T7, and bacteriophage φX174.

6. The engineered multivalent, multiplexed bacteriophage of claim 1, further comprising a third fusion coat protein that includes a third exogenous polypeptide fused to a third bacteriophage coat protein, the third exogenous polypeptide comprising a second targeting polypeptide that includes a second binding sequence that binds a second cancer cell surface antigen.

7. The engineered multivalent, multiplexed bacteriophage of claim 1, further comprising a second, different cancer therapy.

8. The engineered multivalent, multiplexed bacteriophage of claim 1, wherein the first cancer therapy is selected from the group consisting of a toxin, an inhibitor, a drug, and a radioactive metal.

9. The engineered multivalent, multiplexed bacteriophage of claim 8, wherein the first cancer therapy comprises a lactic dehydrogenase inhibitor.

10. The engineered multivalent, multiplexed bacteriophage of claim 1, wherein the first cancer therapy is selected from the group consisting of a cytotoxic agent, a cytostatic agent, an anti-angiogenic agent, a debulking agent, a chemotherapeutic agent, a radiotherapeutic agent, a biological response modifier, a cancer vaccine, a cytokine, a hormone therapy, an oligonucleotide, an antisense nucleotide, an siRNA, and an anti-metastatic agent.

11. The engineered multivalent, multiplexed bacteriophage of claim 1, wherein the first cancer therapy comprises a chelator and a radioactive metal.

12. The engineered multivalent, multiplexed bacteriophage of claim 1, wherein the first cancer cell surface antigen is associated with a solid tumor or a hematological tumor.

13. The engineered multivalent, multiplexed bacteriophage of claim 1, further comprising a fifth fusion coat protein that includes an Interleukin 10 protein or a fragment thereof.

14. The engineered multivalent, multiplexed bacteriophage of claim 1, further comprising a sixth fusion coat protein that is directly or indirectly bonded to a detectable label.

15. A therapeutic composition comprising the engineered multivalent, multiplexed bacteriophage of claim 1 and a delivery system.

16. The therapeutic composition of claim 15, wherein the delivery system is configured to be delivered via an intramuscular, intravenous, subcutaneous, intradermal, or inhalation route.

17. A method for forming an engineered multivalent, multiplexed bacteriophage comprising:
transfecting a bacterial cell with one or more expression plasmids, the one or more expression plasmids comprising a first hybrid nucleic acid sequence including a sequence that encodes a first bacteriophage coat protein ligated to and in frame with a sequence that encodes a targeting polypeptide, the one or more expression plasmids further comprising a second hybrid nucleic acid sequence including a sequence that encodes a second bacteriophage coat protein ligated to and in frame with a sequence that encodes a cancer therapy and/or a sequence that encodes a polypeptide linker, the one or more expression plasmids comprising regulatory sequences such that first and second fusion coat proteins encoded by the first and second hybrid nucleic acid sequences are transiently expressed by the bacterial cell following the transfection; and
infecting the bacterial cell with a bacteriophage; wherein upon the transfection and the infection, the engineered multivalent, multiplexed bacteriophage is produced by the bacterial cell, the engineered multivalent, multiplexed bacteriophage including the first and second fusion coat proteins at a surface thereof.

18. The method of claim 17, wherein the second hybrid nucleic acid sequence encodes the polypeptide linker, and does not encode the cancer therapy, the method further comprising conjugating the cancer therapy to the polypeptide linker of the engineered multivalent, multiplexed bacteriophage.

19. The method of claim 17, wherein the first hybrid nucleic acid sequence is a component of a first expression plasmid and the second hybrid nucleic acid sequence is a component of a second expression plasmid.

20. The method of claim 17, wherein the regulator sequences comprise a first promoter driving expression of the first fusion coat protein and a second promoter driving expression of the second fusion coat protein, and wherein the first promoter and the second promoter are independently selected from an inducible promoter and a native phage promoter.
